(19) United States
(12) Patent Application Publication
(10) Pub. No.: US 2026/0295111 A1
(43) Pub. Date: Oct. 1, 2026

(54) ANTIMICROBIAL COMPOSITIONS AND SUTURES COATED THEREWITH

(71) Applicant: Covidien LP, Mansfield, MA (US)

(72) Inventors: Banu Akar, Cheshire, CT (US); John Michael Marini, Milford, CT (US); Daniel Ciro Broom, Branford, CT (US)

(73) Assignee: Covidien LP, Mansfield, MA (US)

(21) Appl. No.: 19/477,087

(22) PCT Filed: Apr. 15, 2024
(86) PCT No.: PCT/IB2024/053683

§ 371 Date: Oct. 20, 2025

Related U.S. Application Data
(60) Provisional application No. 63/460,890, filed on Apr. 20, 2023.

(51) Int. Cl. A61L 17/00; A61B 17/00; A61B 17/06; A61L 17/12

(52) U.S. Cl. CPC A61L 17/005; A61B 17/06166; A61L 17/12; A61B 2017/00889; A61L 2300/406; A61L 2300/606

(57) ABSTRACT

The present disclosure is directed to antimicrobial compositions for sutures and sutures coated therewith.

### CROSS-REFERENCE TO RELATED APPLICATION

This application claims the benefit of U.S. Provisional Patent Application Ser. No. 63/460,890, filed Apr. 20, 2023, the entire content of which is incorporated herein by reference.

### TECHNICAL FIELD

The present disclosure relates to antimicrobial compositions and antimicrobial sutures coated with the antimicrobial compositions.

### BACKGROUND

Generally, antimicrobial sutures are known in the art. Some examples may include Neosorb® Plus sutures (chlorhexidine diacetate coated PGLA910 and commercially available from Samyang Biopharm) and Vicryl® Plus sutures (triclosan coated copolymer of glycolide and lactide and commercially available from Ethicon, Inc.). However, known antimicrobial sutures may not provide the same knot-tying performance characteristics of ordinary sutures. Known antimicrobial sutures also may not provide protection against microorganisms, and particularly resistant microorganisms, for a sufficient length of time post implantation. For example, some known antimicrobial sutures may only provide an antimicrobial effect for a brief period of time, ranging from a few hours up to a couple of days post implantation. In addition, known antimicrobial sutures, particularly including Triclosan, may be associated with the additional risks of producing microbial resistance, increasing susceptibility to allergies, and/or exposure to one or more carcinogens.

There exists a need for antimicrobial sutures that can provide sufficient and/or extended periods of time with enhanced antimicrobial efficacy in vivo. There is also a need for antimicrobial sutures useful against a broad range of organisms, particularly both gram positive and gram negative organisms, as well as viruses, fungi, and resistant organisms. There is a need for antimicrobial sutures unassociated with furthering microorganism resistance, increasing susceptibility of allergies, and/or including carcinogenic compounds. There is also a need to provide antimicrobial sutures which provide a therapeutic antimicrobial effect, both in the short term and over a longer term, without affecting the performance of the suture.

### SUMMARY

The present disclosure relates to antimicrobial compositions for surgical sutures and sutures coated at least in part with the antimicrobial compositions. The antimicrobial compositions, and/or any coatings formed therefrom, include at least a bioabsorbable polymer derived from one or more lactone monomers, a fatty acid ester, a rifamycin agent, and a tetracycline agent.

In some embodiments, the antimicrobial coating composition includes a bioabsorbable polymer derived from one or more lactone monomers selected the group consisting of lactide, glycolide, trimethylene carbonate, caprolactone, or combinations thereof, and a fatty acid ester which is a lactylate ester of a C₁₀ or greater fatty acid, such as calcium stearoyl lactylate.

In some embodiments, the antimicrobial coating composition includes a rifamycin agent which includes rifampin, and/or a tetracycline agent which includes minocycline.

The antimicrobial coating compositions may further include one or more solvents. In some embodiments, the composition includes one or more, and in some instance all three, solvents selected from the group consisting of methylene chloride, ethanol, hexane, and combinations thereof. The rifampin and/or the minocycline may each represent about 5-7% w/v of the antimicrobial composition including any solvents.

The present disclosure also relates to antimicrobial surgical sutures including a suture made of a biocompatible suture material, and an antimicrobial coating covering at least a portion of the suture, the antimicrobial coating including a bioabsorbable polymer derived from one or more first lactone monomers, a fatty acid ester, and an effective amount of a rifamycin agent and a tetracycline agent. In some embodiments, the suture is a bioabsorbable multifilament suture.

In some embodiments, the antimicrobial coating includes a bioabsorbable polymer derived from one or more first lactone monomers selected the group consisting of lactide, glycolide, trimethylene carbonate, caprolactone, or combinations thereof, and a fatty acid ester which is a lactylate ester of a C₁₀ or greater fatty acid, such as calcium stearoyl lactylate.

In some embodiments, the antimicrobial coating includes a rifamycin agent which includes rifampin, and/or a tetracycline agent which includes minocycline. In some embodiments, a ratio of rifamycin agent to tetracycline agent may be about 1:1 within the coating. In some embodiments, a ratio of rifampin to minocycline is about 1:1 within the coating.

In some embodiments, the biocompatible suture material includes a bioabsorbable suture material including a polymer derived from one or more second lactone monomers.

In some embodiments, the antimicrobial surgical sutures described herein include an absorbable suture made of an absorbable suture material derived from the one or more second lactone monomers selected the group consisting of lactide, glycolide, trimethylene carbonate, caprolactone, or combinations thereof.

In some embodiments, the antimicrobial surgical sutures described herein include a bioabsorbable suture made of a bioabsorbable suture material derived from lactide, glycolide, or combination thereof and an antimicrobial coating including a bioabsorbable polymer derived from caprolactone, glycolide or combination thereof, calcium stearoyl lactylate, rifampin and minocycline. The rifampin and minocycline may be present in a predominant amount of the coating and the bioabsorbable polymer and the calcium stearoyl lactylate may represent a minor amount of the coating. In some embodiments, the coating may include an efficient amount of rifampin and the minocycline suitable for sustained and/or extended release of the agents over 2 or more weeks and particularly 3 weeks.

In some embodiments, the sterile antimicrobial coated sutures described herein may release a rifamycin agent such as rifampin in an original and/or alkylated form for about 4 days, while the release of a tetracycline agent such as minocycline may sustained and/or extended for about 21 days.

Methods of forming and/or using the antimicrobial compositions or the antimicrobial sutures described herein are also provided.

### BRIEF DESCRIPTION OF THE DRAWINGS

The accompanying drawings, which are incorporated in and constitute a part of the specification, illustrate embodiments of the disclosure and, together with a general description of the disclosure given above, and the detailed description of the embodiments given below, serve to explain the principles of the disclosure.

FIGS. 1 and 2 are each a perspective view of a coated suture as described in at least one embodiment herein.

FIG. 3 is a schematic view of a suture coating system and process as described in at least one embodiment herein.

FIG. 4 is a bar graph illustrating a release characteristics of an antimicrobial coated suture as described in at least one embodiment herein;

FIGS. 5A-5C are each an interval plot graph illustrating zone of inhibition data as described in at least one embodiment herein;

FIGS. 6A and 6B include images and a plot graph, respectively, illustrating zone of inhibition data as described in at least one embodiment herein;

FIGS. 7A-7B are each a bar graph illustrating antimicrobial activity data against S. aureus as described in at least one embodiment herein;

FIGS. 8A-8B are each a bar graph illustrating antimicrobial activity data against E. coli as described in at least one embodiment herein;

FIGS. 9A-9B are each a bar graph illustrating antimicrobial activity data against MRSA as described in at least one embodiment herein; and,

FIGS. 10A-10B are each an interval plot graph illustrating mean diameter and knot pull force, respectively, as described in at least one embodiment herein.

### DETAILED DESCRIPTION

The present disclosure relates to antimicrobial compositions and sutures coated with the antimicrobial compositions.

The antimicrobial compositions described herein are designed to be applied to a surgical fastener, such as a surgical suture. The antimicrobial compositions may include at least a bioabsorbable polymer derived from one or more lactone monomers, a fatty acid ester, a rifamycin agent, and a tetracycline agent. One or more solvents and/or carriers may also be included.

The bioabsorbable polymer of the antimicrobial composition may be derived and/or prepared from one or more lactone monomers. By derived, the one or more lactone monomers may be polymerized, alone or with other materials, using any suitable technique to form the bioabsorbable polymer of the antimicrobial compositions. Some non-limiting examples of suitable lactone monomers include lactide (including L-lactide, D-lactide, etc.), glycolide, trimethylene carbonate, tetramethylene carbonate, dioxanones, dioxepanones, caprolactone, valerolactone, and any combinations thereof. In some embodiments, the bioabsorbable polymer of the antimicrobial coating composition is derived only from one or more lactone monomers.

The bioabsorbable polymer of the antimicrobial coating composition may be a homopolymer or copolymer. The copolymer may be a block copolymer, random copolymer, or triblock copolymer. The bioabsorbable polymer may be a linear polymer or a branched polymer.

In some embodiments, the bioabsorbable polymer of the composition may derived from at least two lactone monomers selected from lactide, glycolide, trimethylene carbonate, or caprolactone. In some embodiments, the bioabsorbable polymer may be derived from at least one of glycolide, caprolactone, or combinations thereof. In some embodiments, the polymer may be derived from glycolide and caprolactone, wherein caprolactone represents a predominant amount as compared to the glycolide.

In some embodiments, the bioabsorbable polymer of the composition is a generally linear copolymer derived from polymerization of at least two lactone monomers selected from lactide, glycolide, trimethylene carbonate, or caprolactone. In some embodiments, the bioabsorbable polymer is a generally linear copolymer derived from polymerization of at least glycolide and caprolactone.

In addition to the bioabsorbable polymer derived from at least one monomer, the antimicrobial compositions described herein also include a fatty acid ester salt. The fatty acid ester salt may represent more of the antimicrobial composition than the bioabsorbable polymer (w/v %). Suitable non-limiting examples of fatty acid ester salts include a lactylate ester of a C₁₀ or greater fatty acid, such as magnesium stearoyl lactylate, aluminum stearoyl lactylate, barium stearoyl lactylate zinc stearoyl lactylate, calcium palmityl lactylate, magnesium palmityl lactylate, aluminum palmityl lactylate barium palmityl lactylate, or zinc palmityl lactylate, calcium olelyl lactylate, magnesium olelyl lactylate, aluminum olelyl lactylate, barium olelyl lactylate, zinc olelyl lactylate, calcium stearoyl lactylate, or combinations thereof. In some embodiments, the fatty acid ester salt of the antimicrobial composition is calcium stearoyl lactylate.

The antimicrobial coating compositions described herein also include a rifamycin agent and a tetracycline agent. Suitable non-limiting examples of a rifamycin agent may include rifampin, rifabutin, rifapentine, rifalazil, rifaximin, or any combination thereof. Suitable non-limiting examples of a tetracycline agent may include minocycline, doxycycline, tigercycline, eravacycline, sarecycline, omadacycline, or any combinations thereof. In some embodiments, the rifamycin agent is rifampin and/or the tetracycline agent is minocycline.

The rifamycin agent and the tetracycline agent collectively may represent a “predominant component” of the antimicrobial compositions, and/or any coatings formed therefrom. The bioabsorbable polymer derived from one or more lactone monomers and the fatty acid ester, collectively, may represent a “minor component” of the antimicrobial compositions, and/or any coatings formed therefrom. A “predominant component” represents an amount greater than about 50 weight percent of the components. A “minor component” is a component which is present in an amount up to about 50 weight percent of the components.

In some embodiments, the rifamycin agent and the tetracycline agent, individually or collectively, may represent from about 51 to 75 weight percent of the components and the bioabsorbable polymer and the fatty acid ester collectively may represent from about 25 to 49 weight percent of the components.

In some embodiments, the rifamycin agent and the tetracycline agent may be the only therapeutic or bioactive agents in the antimicrobial composition and/or the antimicrobial suture coating formed therefrom.

In some embodiments, rifampin and minocycline are the only therapeutic or bioactive agents in the antimicrobial composition and/or the antimicrobial suture coating formed therefrom.

In some embodiments, the antimicrobial coating composition, and/or the antimicrobial suture coating formed therefrom, is free of tyrosine, a tyrosine polymer, and/or a tyrosine derivative.

In some embodiments, the antimicrobial composition, and/or the antimicrobial suture coating formed therefrom, includes a bioabsorbable copolymer derived from glycolide and caprolactone, calcium stearoyl lactylate, rifampin, and minocycline.

The rifamycin and tetracycline agents may individually represent from about 2.5 to about 10 percent weight to volume (w/v) of the antimicrobial coating composition. In some embodiments, the rifamycin and tetracycline agents may individually represent from about 5 to about 7.5 percent w/v of the antimicrobial coating composition.

In some embodiments, the antimicrobial coating composition may include rifampin and minocycline representing from about 2.5 to about 10 percent w/v of the coating composition, collectively. In some embodiments, the antimicrobial coating composition may include rifampin and minocycline representing from about 5 to about 7.5 percent w/v of the coating composition, collectively.

The ratio of the bioabsorbable polymer and each of the rifamycin (e.g., rifampin) or the tetracycline (e.g., minocycline) agents in the coating composition may vary depending upon the specific components selected and the particular suture being coated. In some embodiments, the ratio of polymer to rifamycin agent (e.g., rifampin) may be within the range of 1:1.1 to 1:4 parts by weight and in some embodiments within the range of 1:1.5 to 1:3 parts by weight. In some embodiments, the ratio of polymer to tetracycline agent (e.g., minocycline) may be within the range of 1.1:1 to 1:4 parts by weight and in some embodiments within the range of 1:1.5 to 1:3 parts by weight.

The antimicrobial compositions described herein may be formed by combining (e.g., mixing or blending) the one or more bioabsorbable polymers derived from lactone monomers, the one or more fatty acid ester salts, the rifamycin agent, and the tetracycline agent in one or more solvents. The combining of the coating ingredients can be done in a single step or multiple steps (or pots).

In some embodiments, the one or more bioabsorbable polymers derived from lactone monomers, the one or more fatty acid ester salts, and one or more solvents may be combined to form an initial mixture or solution and then the rifamycin agent and the tetracycline agent may be added to the initial mixture or solution to form a second solution suitable as an antimicrobial coating composition as described herein.

Suitable non-limiting examples of solvents include methylene chloride, ethanol, hexanes, acetones, or any combination thereof. In some embodiments, the antimicrobial coating composition includes three solvents including methylene chloride, ethanol, and hexanes. In some embodiments, the antimicrobial coating composition includes about 60 to 80 percent by volume methylene chloride, about 10 to about 20 percent by volume ethanol, and about 5 to about 20 percent by volume hexanes.

In some embodiments, the antimicrobial composition includes a bioabsorbable copolymer derived from one or more lactone monomers, a fatty acid ester, a rifamycin agent, a tetracycline agent, and two or more solvents selected from methylene chloride, ethanol, and hexanes.

In some embodiments, the antimicrobial composition includes a bioabsorbable copolymer derived from glycolide and caprolactone, calcium stearoyl lactylate, rifampin, minocycline, and three solvents including methylene chloride, ethanol, and hexanes.

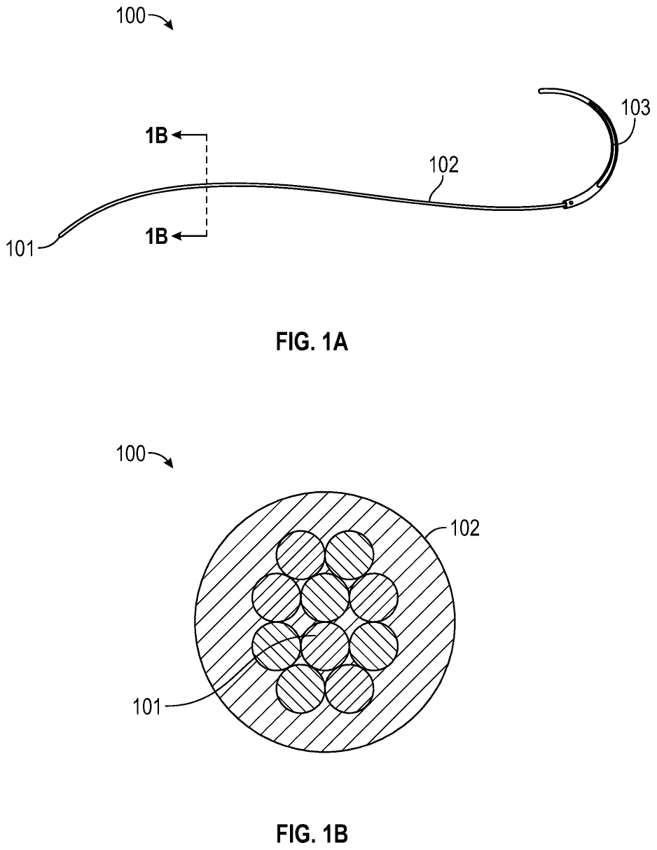

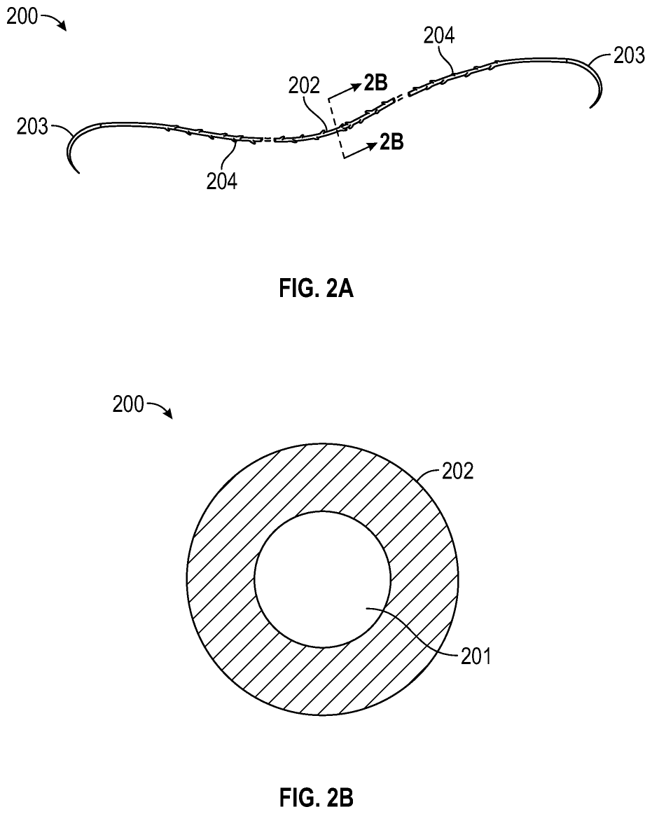

As shown in FIGS. 1A-2B, sutures including the antimicrobial coating compositions described herein are also provided. Specifically, FIGS. 1A and 1B depict an antimicrobial coated suture 100 including a suture 101 and an antimicrobial coating 102. Suture 101 is depicted as a multifilament suture. The antimicrobial coating 102 is depicted covering and/or coating at least a portion of the multifilament suture 101. The antimicrobial coating 102 may cover any amount and/or portion, if not all, of the suture 101. The antimicrobial suture 100 may include a surgical needle 103 on at least one end thereof.

FIGS. 2A and 2B depict a coated suture 200 including a suture 201 and an antimicrobial coating 202. Suture 201 is depicted as a monofilament suture including barbs 204. The antimicrobial coating 202 is depicted covering and/or coating at least a portion of the monofilament suture 201. The antimicrobial coating 202 may cover any amount and/or portion, if not all, of the suture 201 and/or barbs 204. The antimicrobial suture 200 may include a surgical needle 203 on at least one end thereof.

FIG. 2A also depicts the barbs 204 as opposing bidirectional barbs, as may be common with a double-armed suture as depicted. However, in some embodiments, the barbs may be unidirectional barbs, and particularly when the suture is either free of a surgical needle or armed with at least one surgical needle as shown in FIG. 1A.

The sutures described herein may be monofilament or multi-filament sutures. The sutures may include barbs or be barb free. The sutures described herein may be formed from any sterilizable biocompatible material including bioabsorbable polymeric materials, non-bioabsorbable polymeric materials, or both.

Some non-limiting examples of suitable bioabsorbable suture materials include polymers derived and/or prepared from lactone monomers such as lactide (including L-lactide, D-lactide, etc.), glycolide, trimethylene carbonate, tetramethylene carbonate, dioxanones, dioxepanones, caprolactone, valerolactone, or any combinations thereof; biopolymers derived from proteins such as collagen (I, II and III), elastin, fibrin, fibrinogen, silk, and/or albumin; polysaccharides such as hyaluronic acid (HA), dextran, alginate, chitin, chitosan, and/or cellulose; and/or catgut.

Some non-limiting examples of suitable non-bioabsorbable suture materials polyolefins such as polyethylene (including ultra-high molecular weight polyethylene) and polypropylene including atactic, isotactic, syndiotactic, and blends thereof; polyethylene glycols; polyethylene oxides; ultra-high molecular weight polyethylene; copolymers of polyethylene and polypropylene; polyisobutylene and ethylene-alpha olefin copolymers; fluorinated polyolefins such as fluoroethylenes, fluoropropylenes, fluoroPEGSs, and polytetrafluoroethylene; polyamides such as nylon, Nylon 6, Nylon 6,6, Nylon 6,10, Nylon 11, Nylon 12; polyesters such as polyethylene terephthalate, polyethylene naphthalate, polytrimethylene terephthalate, and polybutylene terephthalate; polyethers; polybutester; polytetramethylene ether glycol; 1,4-butanediol; and/or polyurethanes.

The sutures described herein may be formed using any technique within the purview of those skilled in the art, such as, for example, extrusion, molding, casting and/or spinning. Where the suture is made of multifilament suture, the filaments may be combined using any known technique such as, for example, braiding, weaving, or knitting. The sutures may also be drawn, oriented, annealed, calendared, crinkled, twisted, commingled, or air entangled, as part of the suture forming process. In one embodiment, a multifilament suture may be produced by braiding. The braiding may be done by any method within the purview of those skilled in the art.

It is contemplated that the sutures and/or antimicrobial compositions described herein may include one or more dyes to increase visibility if used in the surgical field. Any dyes known to be suitable for incorporation in sutures can be used. Such dyes include but are not limited to carbon black, bone black, D&C Green No. 6, and D&C Violet No. 2 as described in the handbook of U.S. Colorants for Food, Drugs and Cosmetics by Daniel M. Marrion (1979).

In some embodiments, the sutures described herein may be an absorbable multifilament suture made of a suture bioabsorbable polymer and coated at least in part with an antimicrobial coating including a coating bioabsorbable polymer wherein each of the suture and coating bioabsorbable polymers may be derived from the same or different lactone monomer(s), such as, for example, lactide, glycolide, caprolactone, trimethylene carbonate, or combinations thereof.

The antimicrobial coatings described herein are derived and/or prepared from the antimicrobial coating compositions as further provided herein.

The antimicrobial coating compositions can be applied to a suture by any suitable process. Some non-limiting examples include dip-coating, brush-coating, spray-coating, roll-coating, and the like. In some embodiments, the sutures described herein may be dip-coated with the antimicrobial compositions.

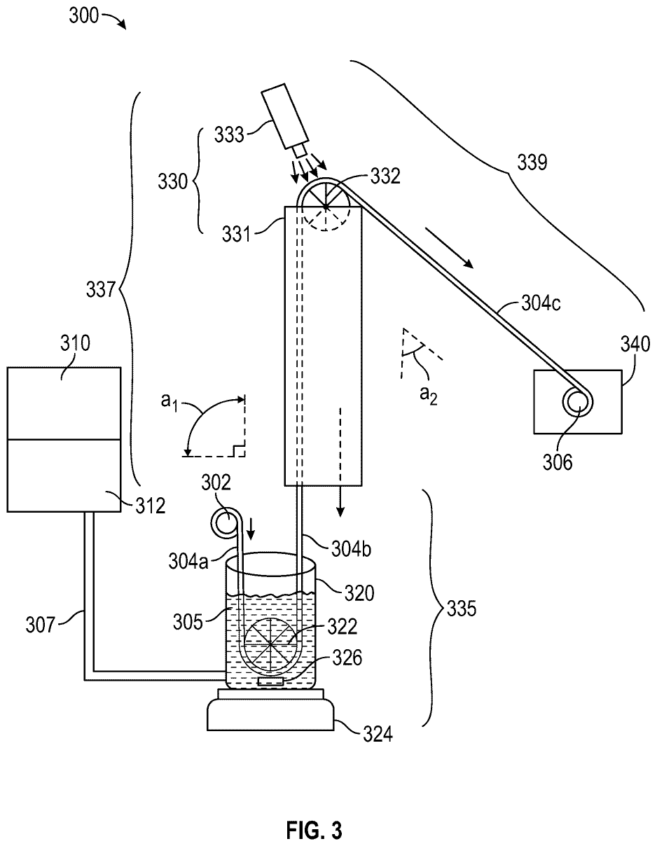

FIG. 3 further depicts a coating system 300 used in methods of forming an antimicrobial coated suture including dip-coating. The coating system 300 may include one or more of a first spool 302 of an uncoated suture 304a, a second spool 306 for receiving a coated suture 304c, a control panel 310, a closed container 312 configured to receive an antimicrobial composition 305 in bulk (FIG. 3B), an open container 320 configured to receive the antimicrobial composition 305 in smaller amounts sufficient for a given coating process, a nozzle 307 configured to control the transfer the composition 305 from the closed container 312 to the open container 320, a first wheel 322, a balance 324, a stirrer 326, a drying device 330, a second wheel 332, and/or a feeder machine 340.

The control panel 310 may be configured to control any part of the coating system 300, individually or collectively. For example, in some embodiments, the control panel 310 may control the transfer of the antimicrobial composition 305 from the closed container 312 to the open container 320, the speed by which the feeder machine 340 draws the suture 304 through the coating system 300, the temperature and/or air flow of the drying device 330, the stirring and temperature of the composition 305 inside of either of the closed or open containers 312, 320, or any combination thereof.

In the methods of forming a coated antimicrobial suture, as further illustrated in FIG. 3, the feeder machine 340 passes and/or draws one or more uncoated sutures 304a from a first spool 302 to a second spool 306 (as a coated antimicrobial suture 304c) wherein the uncoated suture 304a is dip-coated into a coating composition 305 and dried therebetween.

Initially, step 335, the machine 340 may draw one or more uncoated sutures 304a on a first spool 302 around a first wheel 322 and through an open container 320, e.g., beaker or vat, including an antimicrobial coating composition 305 thereby forming a wet suture covered with the antimicrobial composition 304b. At least a portion, if not all, of the first wheel 322 maybe positioned within the container 320 and/or submerged in the composition 305. The composition 305 may be under constant or intermittent stirring via magnetic stirrer 326 and balance 324 upon which open container 320 may sit.

The feed machine 340 may draw the uncoated suture through the system at a digital output speed ranging from 1-5 m/min. In some embodiments, the digital output speed may range from about 2-4 m/min. At these output speeds, the dwell time of the suture in the antimicrobial coating composition may range from 1 to 5 seconds or 2 to 4 seconds, respectively.

Next, in step 337, the machine 340 continues to draw and/or pass the wet suture 304b vertically upward from the container 320 (and/or the first wheel 322) and through a vertical drying device 330, such as an oven or heated tunnel, to one or more second wheels 332 thereby forming a generally dry antimicrobial coating on the suture 310c with a predominant amount, if not all, of any solvent of the antimicrobial composition driven off to form the antimicrobial coating. At least a portion, if not the majority, of the second wheel 332 may be positioned within the drying device 330.

In some embodiments, the vertical upward path of the wet suture 304b between the first and second wheels 322, 332 forms a first angle a₁ relative to the first spool 302 which may range between 70 to 110 degrees. In some embodiments, the first angle a₁ may range between 80 to 100 degrees. In some embodiments, the first angle a₁ may be about 90 degrees.

The drying device 330 may be configured to heat the air therein to a temperature ranging from 50 to 150° C. In some embodiments, the temperature may range from 75 to 125° C. In some embodiments, the temperature may be about 100° C.

The drying device 330 may also be configured to create a flow of the heated air therein vertically downward through the drying device 330 towards the open container 320. For example, as shown, in some embodiments, the drying device 330 may include a generally open vertical tube or tunnel 331 through which heated air (multiple downward arrows) is blown by a separate hot air gun 333. The separate air gun 333 may be positioned near and/or above the second wheel 332. In another example, the drying device may be a generally closed oven including a fan configured to move the heated air, such as or similar to a convection oven.

The dwell time of the coated suture inside the drying device 330 can range from about 60 seconds to about 600 seconds. In some embodiments, the dwell time can be controlled by the number of second wheels associated with the drying device. For example, in some embodiments, the system may include two or more wheels positioned within the drying device upon which the coated suture must pass while inside the drying device thereby extending the dwell time therein. In some embodiments, each internal wheel may increase the dwell time by about 60 seconds. For example, a system including two (2) or three (3) wheels inside the drying device may render the dwell time of the suture inside the device to be from about 120 to 180 seconds.

Next, in step 339, the machine 340 continues to draw and/or pass the suture 304c vertically and horizontally downward from the second wheel 332 (and/or the exiting end of the drying device 330) at a second draw angle a₂ to the second spool 306. Additional drying, if needed, may occur at generally room temperature and under some force of gravity as the suture 304c exits downwardly from the drying device 330 towards the second spool 306.

In some embodiments, the second angle a₂ may range from 25 to 65 degrees. In some embodiments, the second angle a₂ may range from 35 to 55 degrees. In some embodiments, second angle a₂ may be about 45 degrees.

In some embodiments, the amount of the rifamycin agent, and particularly rifampin, contained in the coated suture may range from about 0.1-500 μg/mg suture. In some embodiments, the coated antimicrobial suture may include about 1-100 μg/mg suture of the rifamycin agent. In some embodiments, the coated antimicrobial suture may include about 10-75 μg/mg suture of the rifamycin agent. In some embodiments, the coated antimicrobial suture may include about 15-50 μg/mg suture of the rifamycin agent.

The amount of the tetracycline agent, and particularly minocycline, contained in the coated suture may range from about 0.1-500 μg/mg suture. In some embodiments, the coated antimicrobial suture may include about 1-100 μg/mg suture of the tetracycline agent. In some embodiments, the coated antimicrobial suture may include about 10-75 μg/mg suture of the tetracycline agent. In some embodiments, the coated antimicrobial suture may include about 15-50 μg/mg suture of the tetracycline agent.

The coated sutures described herein may be sterilized and packaged using any suitable method. In some embodiments, the antimicrobial coated suture may be sterilized via known ethylene oxide processes.

In some embodiments, following sterilization via ethylene oxide, at least some of the rifamycin agent (e.g., rifampin) may become alkylated (e.g., alkylated rifamycin agent and/or alkylated rifampin) thereby providing a sterile coated suture wherein the coating includes both rifampin and alkylated rifampin, as well as minocycline. In some embodiments, the sterile coating may include 50-70% rifamycin agent (e.g., rifampin) in its original form, i.e., not alkylated, and 30-50% alkylated rifamycin agent (e.g., alkylated rifampin).

In some embodiments, the amount of a rifamycin agent and an alkylated rifamycin agent, and particularly rifampin and alkylated rifampin, contained in the coated suture may range from about 0.1-500 μg/mg suture collectively. In some embodiments, the coated antimicrobial suture may include about 1-100 μg/mg suture of the rifamycin agent and alkylated rifamycin agent collectively. In some embodiments, the coated antimicrobial suture may include about 10-75 μg/mg suture of the rifamycin agent and alkylated rifamycin agent collectively. In some embodiments, the coated antimicrobial suture may include about 15-50 μg/mg suture of the rifamycin agent and alkylated rifamycin agent collectively.

In some embodiments, the coated antimicrobial suture may include about 15-25 μg/mg suture of the rifamycin agent (e.g., rifampin), about 15-25 μg/mg suture of the alkylated rifamycin agent (e.g., alkylated rifampin), and about 15-25 μg/mg suture of the tetracycline agent (e.g., minocycline).

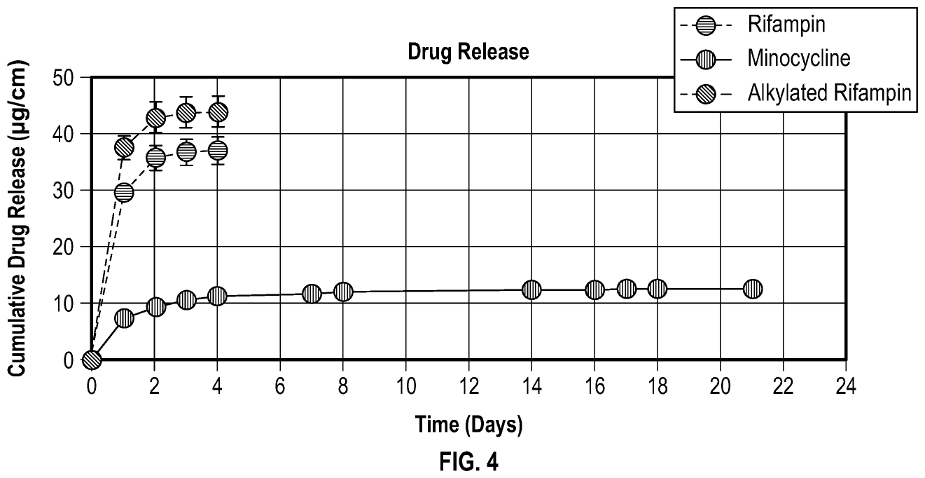

As illustrated in FIG. 4, in some embodiments, the sterile antimicrobial coated sutures described herein may release a rifamycin agent such as rifampin in an original and/or alkylated form over 4 days, while the release of a tetracycline agent such as minocycline may sustained and/or extended for up to 21 days. As further illustrated in FIG. 4, in some embodiments, the sterile antimicrobial coated sutures described herein may provide a cumulative release of between 35 and 45 μg/cm suture of a rifamycin agent such as rifampin in an original and/or alkylated form over 4 days and a cumulative release of between 5 and 10 μg/cm suture of a tetracycline agent such as minocycline sustained and/or extended for up to 21 days.

In some embodiments, the antimicrobial sutures described herein include a sustained release antimicrobial coating releasing the rifamycin agent and the tetracycline agent over 4 days and 21 days, respectively.

The antimicrobial sutures described herein may also be used for closing a wound in tissue. Such methods may include passing an antimicrobial coated suture including a needle on at least one end thereof through tissue to close the wound, the antimicrobial coated suture including a suture made of biocompatible material and an antimicrobial coating including a bioabsorbable polymer derived from one or more first lactone monomers, a fatty acid ester, a rifamycin agent, and a tetracycline agent, and optionally tying a knot. The suture coating is derived from any of the antimicrobial coating compositions described herein.

The invention may be further described by reference to the following numbered paragraphs:

1. An antimicrobial coating composition for a surgical suture comprising:
a bioabsorbable polymer derived from one or more lactone monomers,
a fatty acid ester,
a rifamycin agent, and
a tetracycline agent.

2. The antimicrobial coating composition of numbered paragraph 1, wherein the bioabsorbable polymer is derived from one or more lactone monomers selected from the group consisting of lactide, glycolide, trimethylene carbonate, caprolactone, and combinations thereof.

3. The antimicrobial coating composition of numbered paragraph 1, wherein the bioabsorbable polymer derived from one or more lactone monomers includes a bioabsorbable copolymer derived include caprolactone and glycolide, wherein caprolactone represents a predominant amount of the bioabsorbable polymer.

4. The antimicrobial coating composition of numbered paragraph 1, wherein the fatty acid ester is calcium stearoyl lactylate.

5. The antimicrobial coating composition of numbered paragraph 1, wherein the rifamycin agent is rifampin and the tetracycline agent is minocycline.

6. The antimicrobial coating composition of numbered paragraph 5, wherein the rifampin and the minocycline collectively represent a predominant component of the composition and the bioabsorbable polymer and the fatty acid ester collectively represent a minor component of the composition.

7. The antimicrobial coating composition of numbered paragraph 1, further comprising one or more solvents selected from the group consisting of methylene chloride, ethanol, hexane, and combinations thereof.

8. An antimicrobial surgical suture comprising:
a suture made of a biocompatible suture material, and
an antimicrobial coating covering at least a portion of the suture, the antimicrobial coating including a minor amount of a bioabsorbable polymer derived from one or more first lactone monomers and a fatty acid ester, and a predominant amount of a rifamycin agent and a tetracycline agent.

9. The suture of numbered paragraph 8, wherein the suture is a multifilament suture.

10. The suture of numbered paragraph 8, wherein the biocompatible suture material comprises a bioabsorbable suture material including a polymer derived from one or more second lactone monomers.

11. The suture of numbered paragraph 10, wherein the polymer derived from one or more second lactone monomers is a bioabsorbable copolymer derived from lactide and glycolide.

12. The suture of numbered paragraph 11, wherein the bioabsorbable polymer derived from one or more first lactone monomers includes a bioabsorbable copolymer derived from caprolactone and glycolide, wherein caprolactone represents a predominant amount.

13. The suture of numbered paragraph 8, wherein the fatty acid ester is calcium stearoyl lactylate.

14. The suture of numbered paragraph 8, wherein the rifamycin agent is rifampin and the tetracycline agent is minocycline.

15. The suture of numbered paragraph 14, wherein the antimicrobial coating further comprises alkylated rifampin, and the rifampin, the alkylated rifampin, and the minocycline each represent about 15-25 μg/mg of the suture.

16. The suture of numbered paragraph 14, wherein the antimicrobial coating is sterile and includes about 30-50% alkylated rifampin.

17. The suture of numbered paragraph 8, wherein the antimicrobial coating is configured to be a sustained release antimicrobial coating releasing the rifamycin agent and the tetracycline agent over 4 days and 21 days, respectively.

18. The suture of numbered paragraph 8, wherein the antimicrobial coating produces at least a 4 log CFU reduction of S. aureus, E. coli, or MRSA in vivo.

19. A method of suturing a wound comprising:
passing an antimicrobial coated suture including a needle on at least one end of the suture through tissue to close the wound, the antimicrobial coated suture including at least one filament of biocompatible suture material, and an antimicrobial coating including a bioabsorbable polymer derived from one or more first lactone monomers, a fatty acid ester, a rifamycin agent, and a tetracycline agent.

20. A method of forming a coated antimicrobial suture comprising:
drawing an uncoated suture through an open container including an antimicrobial coating composition to form a wet coated suture, the antimicrobial coating composition including a bioabsorbable polymer derived from one or more lactone monomers, a fatty acid ester, a rifamycin agent, a tetracycline agent, and a solvent,
drawing the wet coated suture upwardly at a first angle from the open container and through a drying device to form a dry antimicrobial coated suture, and
drawing the dry coated suture downwardly at a second angle from an exit end of the drying device to a spool.

It will be understood that various modifications may be made to the embodiments of the presently disclosed composite implants. Therefore, the above description should not be construed as limiting, but merely as exemplifications of embodiments. Those skilled in the art will envision other modifications within the scope and spirit of the present disclosure.

### EXAMPLES

### Example 1

An antimicrobial coating composition was prepared by combining the material as provided in Table 1 below.

| | | |
|---|---|---|
| Solids (w/Vt %) | Poly(caprolactone-glycolide) | 2.83 |
| | Calcium stearoyl lactylate | 3.07 |
| | Rifampin | 5.25 |
| | Minocycline | 6.8 |
| Solvents (v/Vt %) | Methylene Chloride | 65 |
| | Ethanol | 19 |
| | Hexanes | 15 |

Vt (volume solvent total): Methylene Chloride + Ethanol + Hexanes

To create the antimicrobial coating composition, a bioabsorbable polymer (e.g. poly(caprolactone-glycolide)), a fatty acid ester salt (e.g., calcium stearoyl lactylate), a rifamycin and tetracycline agent (e.g., rifampin and minocycline) were added to a container on a mass balance, followed by the addition of the solvents in a fume hood. The mixture was then stirred for approximately 5 minutes.

### Example 2

Polysorb® (Medtronic) 2-0 braided multifilament uncoated sutures (both dyed and undyed) were continuously dip-coated with the antimicrobial coating composition of Example 1. The dip-coating system was set up in a walk-in fume hood.

Approximately 300 mLs of the antimicrobial coating composition of Example 1 were meter-pumped from a first closed container to a second open container, such as a beaker, including a magnetic stir bar and positioned on a magnetic stir plate set at 400 rpms.

A cotton leader was fed through the entire suture coating path and attached to a first spool of uncoated Polysorb® 2-0 suture on one end of the coating path and a second spool intended to collect the suture after coating on the opposite end of the coating path. The suture coating path extended from the first spool, wrapping around a first ceramic wheel positioned near the bottom of the container including the antimicrobial coating composition, without contacting or interfering with the magnetic stir bar. The suture path continued from the first wheel, through a tunnel oven vertically aligned with the beaker, to a second wheel positioned on an opposite end of the tunnel oven. The tunnel oven was maintained at a temperature of about 100° C. with nitrogen flow. The second wheel directed the suture path downwardly and away from the tunnel oven to the second spool intended to collect the suture after coating. The second spool was connected to a feed machine designed to draw the suture through the suture path.

The feed machine pulled the cotton leader and Polysorb® suture through the suture coating path (e.g., through the antimicrobial coating composition in the beaker, around the first wheel, vertically through the tunnel oven, around the second wheel, and onto the second spool) at a digital output speed of 2.5 m/min. At this output speed, the dwell time of the suture in the antimicrobial coating composition was approximately 2.5 seconds and the dwell time of the suture in the tunnel oven was approximately _180_seconds. After the Polysorb® suture was coated and captured on the second output spool, the coated suture was allowed to further dry for at least 1 hour in the fume hood before being sterilized via ethylene oxide and packaged.

### Example 3

Zone of Inhibition Assays were performed using modified Kirby-Bauer agar diffusion tests comparing the coated sutures of Example 2, uncoated Polysorb® sutures, and Vicryl® Plus sutures (including Triclosan) individually against S. aureus, E. Coli, and MRSA.

Using an aseptic technique, a sterile swab was placed into a Tryptic Soy Broth culture of a given bacteria (S. aureus, E. Coli, or MRSA) and excess liquid was removed by gently pressing the swab against the inside of the tube. Using the swab, a bacterial lawn was formed by streaking each Tryptic Soy Agar (TSA) plate. Each plate was allowed to dry for approximately 5 minutes.

Several of the each of the coated sutures of Example 2, uncoated Polysorb® sutures, and Vicryl® Plus sutures were passed through a sterile 20% collagen gel to simulate passage through tissue. Samples were either passed through the gel 1× or 10×. Using flame-sterilized forceps, a 2 cm length of each suture was gently pressed onto the agar in the center of the plate to prevent shifting. The plates were then incubated at 37° C. (98.6° F.) for at least 72 hours. Measurement of the zone of inhibition for each sample was performed after 72 hours.

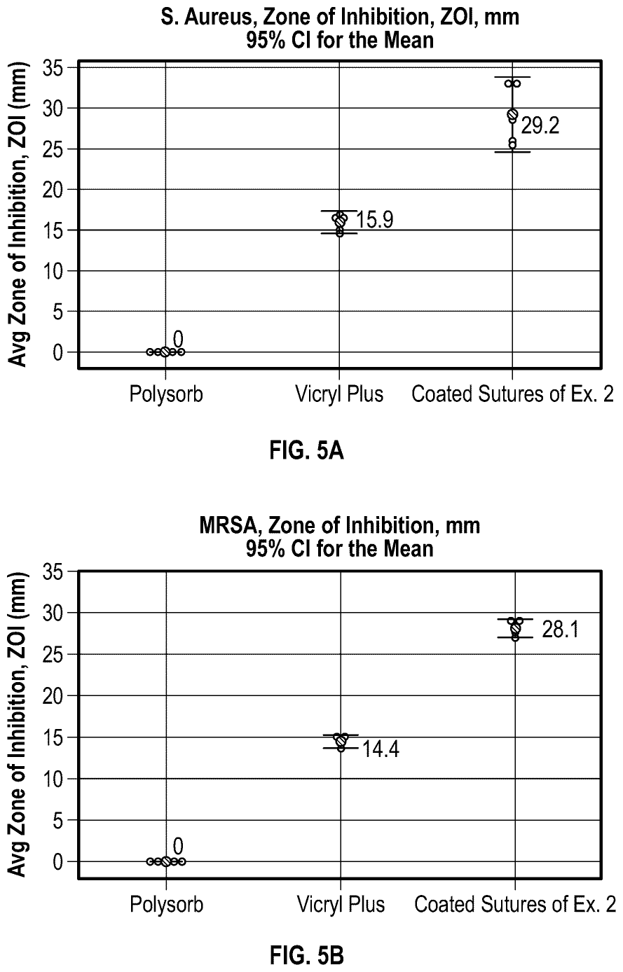

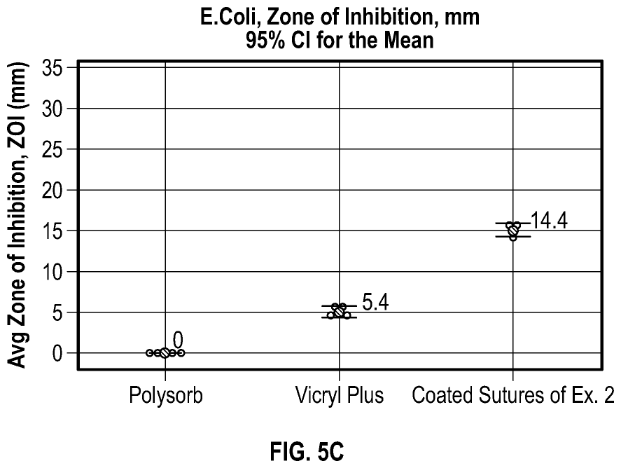

As shown in FIGS. 5A-5C, the coated sutures described herein, and particularly of Example 2, provided an average zone of inhibition in vitro of at least 15 mm for at least 72 hours against each of S. aureus, MRSA, and E. Coli, respectively. As specifically shown in FIG. 5A, the coated sutures described herein, and particularly of Example 2, provided an average zone of inhibition in vitro of at least 25 mm for at least 72 hours against S. aureus. As specifically shown in FIG. 5B, the coated sutures described herein, and particularly of Example 2, provided an average zone of inhibition in vitro of at least 25 mm for at least 72 hours against MRSA.

### Example 4

Zone of Inhibition Assays were performed using modified Kirby-Bauer agar diffusion tests comparing the coated sutures of Example 2 and Vicryl® Plus sutures (including Triclosan) against S. aureus for at least 2 weeks.

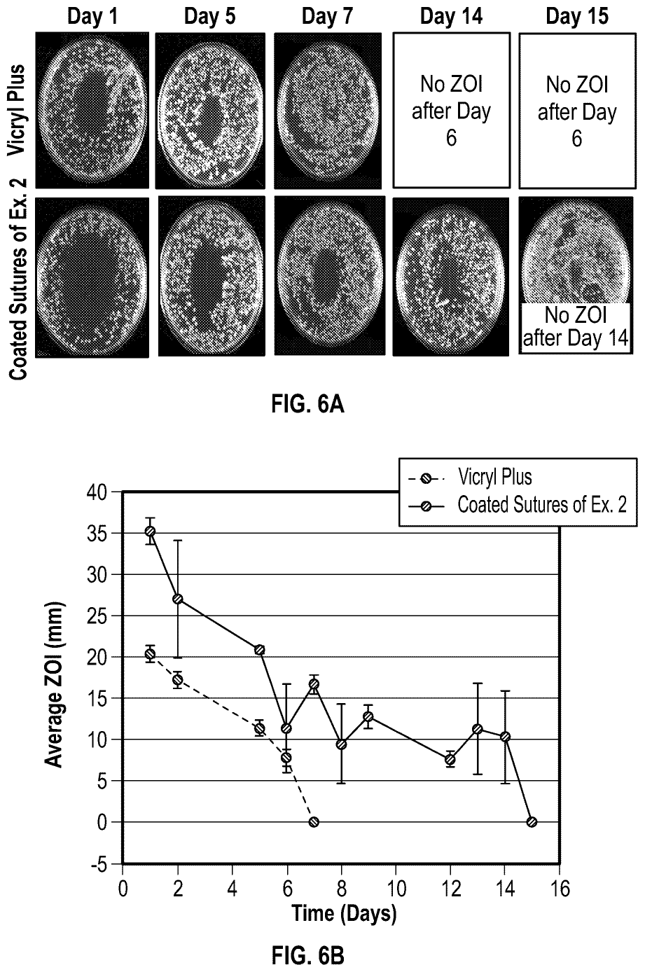

As shown in FIGS. 6A-6B, the coated sutures described herein, and particularly of Example 2, provided a zone of inhibition in vitro for more than a week, and particularly at least two weeks, against S. aureus. While sutures coated with Triclosan (e.g., Vicryl® Plus sutures) only provided a zone of inhibition against S. aureus for less than a week.

In some embodiments, the coated sutures described herein, and particularly of Example 2, provided an in vitro average zone of inhibition of at least 10 mm for at least two weeks against S. aureus.

### Example 5

The antibacterial efficacy in vivo of several of the each of the coated sutures of Example 2, uncoated Polysorb® sutures, Vicryl® Plus sutures (Johnson and Johnson), and Neosorb® Plus sutures (Samyang) were tested against S. aureus.

A 20-gauge catheter was inserted under the clean skin of an anaesthetized rat. A test suture was passed completely through the entire length of the catheter until protruding from both ends of the catheter. A sterile syringe was attached to a hub of the catheter to slowly inject 0.3 ml of S. aureus into the catheter as the catheter was withdrawn and the suture was held in place. A first group of rats received 0.3 ml of 1×10⁵ CFU/ml of S. aureus and a second group of rats received 0.3 ml of 1×10⁶ CFU/ml of S. aureus. Exit holes in the skin from the removed catheter and the exposed remaining suture material ends were closed with a tissue glue and the portions of the suture protruding from the skin were clipped to be flush with the skin.

After 48 hours post-inoculation, the implanted suture was carefully removed from the rat and placed in 2.0 mL of sterile Dey Engley (D/E) Broth and further processed for CFU counts.

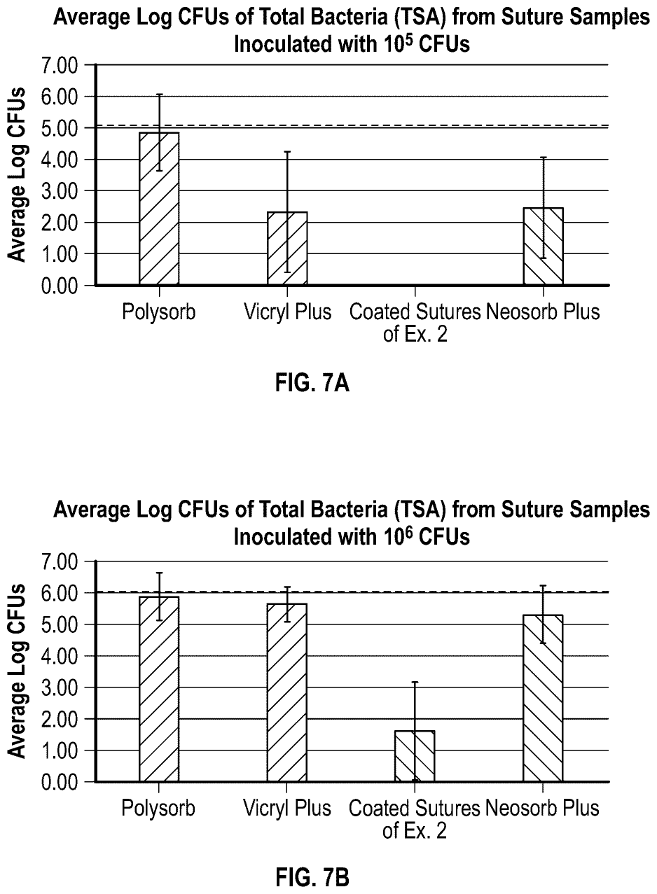

FIG. 7A depicts the total bacteria of the various explanted sutures (t=48 h) of samples infected with S. aureus with a concentration of 10⁵ CFUs/mL. FIG. 7B depicts the total bacteria of the various explanted sutures (t=48 h) of samples infected with S. aureus with a concentration of 10⁶ CFUs/mL. As shown in FIGS. 7A-7B, the coated sutures described herein, and more particularly of Example 2, demonstrated at least a 3 log, and particularly a 4 log or 5 log, reduction against S. aureus for at least 48 hours post implantation.

### Example 6

The antibacterial efficacy in vivo of several of the each of the coated sutures of Example 2, uncoated Polysorb® sutures, and Vicryl® Plus sutures (Johnson and Johnson) were tested against E. coli.

A 20-gauge catheter was inserted under the clean skin of an anaesthetized rat. A test suture was passed completely through the entire length of the catheter until protruding from both ends of the catheter. A sterile syringe was attached to a hub of the catheter to slowly inject 0.3 ml of E. coli into the catheter as the catheter was withdrawn and the suture was held in place. Each of the rats received 0.3 ml of 1×10⁶ CFU/ml of E. coli. Exit holes in the skin from the removed catheter and the exposed remaining suture material ends were closed with a tissue glue and the portions of the suture protruding from the skin were clipped to be flush with the skin.

After 48 hours post-inoculation, the implanted suture was carefully removed from the rat and placed in 2.0 mL of sterile Dey Engley (D/E) Broth and further processed for CFU counts. In addition, a biopsy of tissue was collected adjacent to the site previously covered by the suture. The tissue sample was carefully removed from the rat and placed in 2.0 mL of sterile Dey Engley (D/E) Broth and further processed for CFU counts.

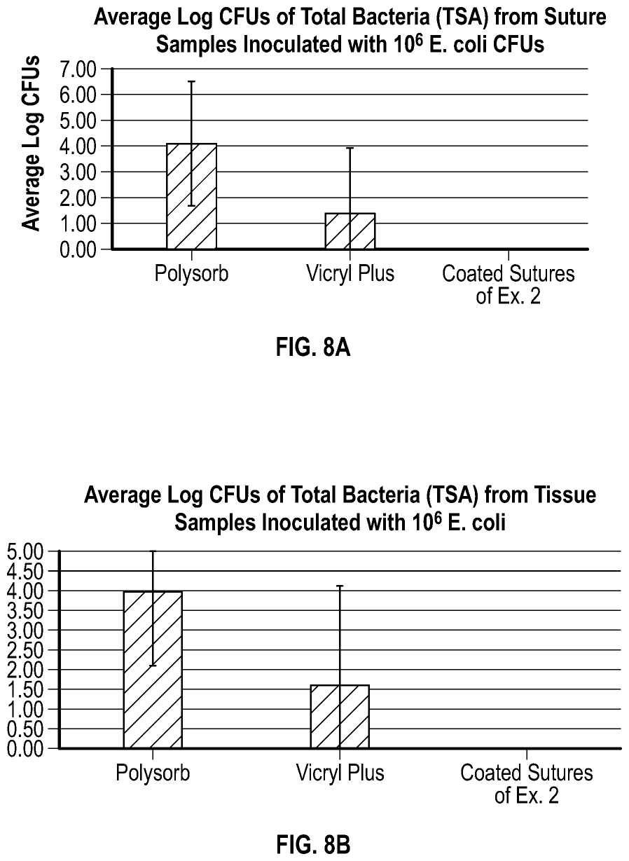

FIG. 8A depicts the total bacteria of the various explanted sutures (t=48 h) of samples infected with E. coli with a concentration of 10⁶ CFUs/mL. FIG. 8B depicts the total bacteria of the various biopsied tissue samples (t=48 h) adjacent to the site of suture infected with E. coli with a concentration of 10⁶ CFUs/mL. As shown in FIGS. 8A-8B, the coated sutures described herein, and more particularly of Example 2, demonstrated at least a 3 log, and particularly a 4 log, 5 log, or 6 log, reduction against E. coli for at least 48 hours post implantation.

### Example 7

The antibacterial efficacy in vivo of several of the each of the coated sutures of Example 2, uncoated Polysorb® sutures, and Vicryl® Plus sutures (Johnson and Johnson) were tested against E. coli.

A 20-gauge catheter was inserted under the clean skin of an anaesthetized rat. A test suture was passed completely through the entire length of the catheter until protruding from both ends of the catheter. A sterile syringe was attached to a hub of the catheter to slowly inject 0.3 ml of MRSA into the catheter as the catheter was withdrawn and the suture was held in place. Each of the rats received 0.3 ml of 1×10⁷ CFU/ml of MRSA. Exit holes in the skin from the removed catheter and the exposed remaining suture material ends were closed with a tissue glue and the portions of the suture protruding from the skin were clipped to be flush with the skin.

After 48 hours post-inoculation, the implanted suture was carefully removed from the rat and placed in 2.0 mL of sterile Dey Engley (D/E) Broth and further processed for CFU counts. In addition, a biopsy of tissue was collected adjacent to the site previously covered by the suture. The tissue sample was carefully removed from the rat and placed in 2.0 mL of sterile Dey Engley (D/E) Broth and further processed for CFU counts.

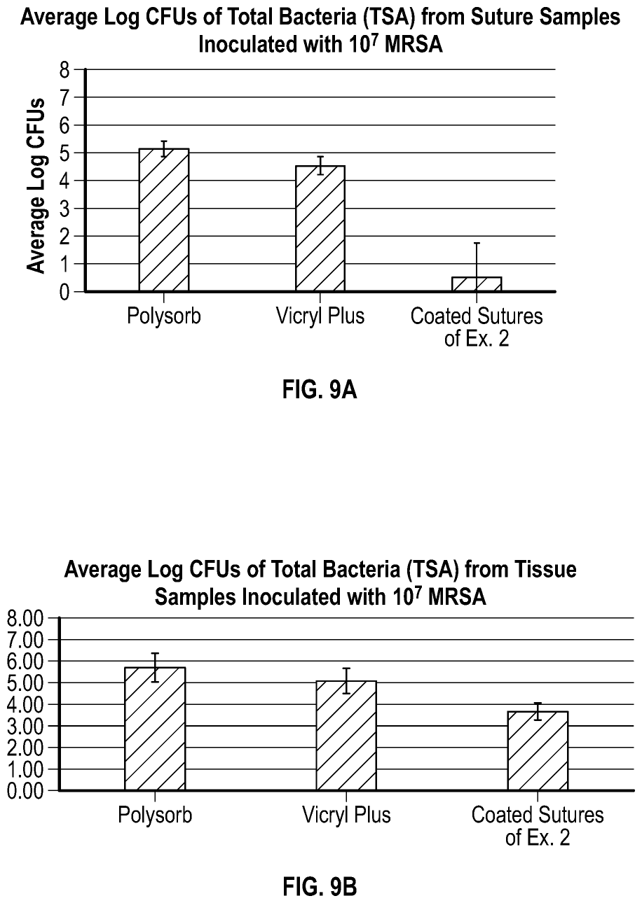

FIG. 9A depicts the total bacteria of the various explanted sutures (t=48 h) of samples infected with MRSA with a concentration of 10⁷ CFUs/mL. FIG. 9B depicts the total bacteria of the various biopsied tissue samples (t=48 h) adjacent to the site of suture infected with MRSA with a concentration of 10⁷ CFUs/mL. As shown in FIGS. 9A-9B, the coated sutures described herein, and more particularly of Example 2, demonstrated at least a 3 log, and particularly a 4 log, 5 log, or 6 log, reduction against MRSA for at least 48 hours post implantation.

### Example 8

The performance of several of the each of the coated sutures of Example 2, uncoated Polysorb® sutures, and Vicryl® Plus sutures (Johnson and Johnson) were also tested according to USP standards.

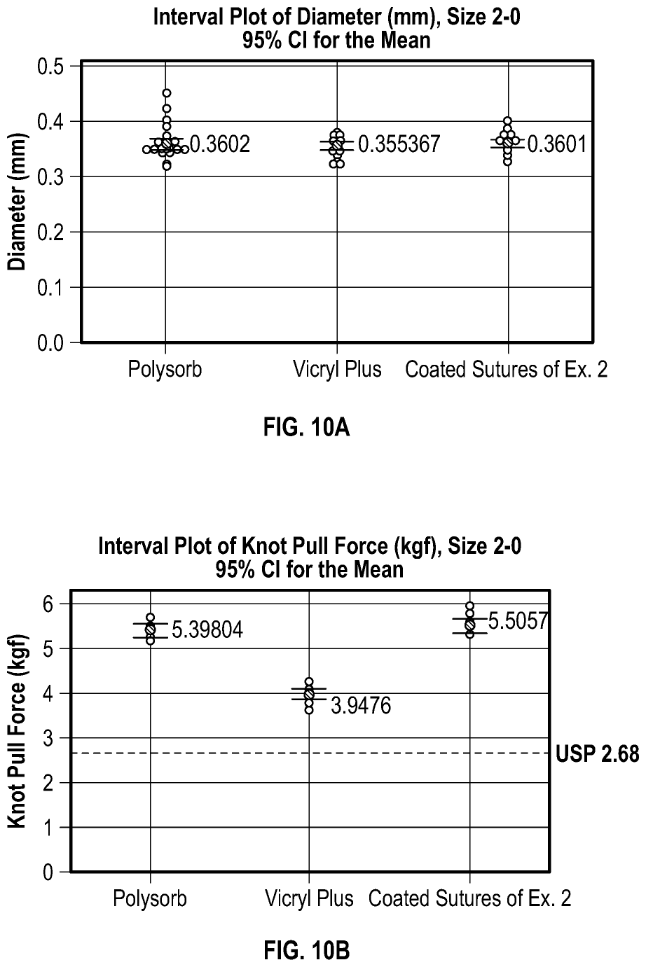

As shown in FIGS. 10A and 10B, the average diameter of the size 2-0 sutures tested are generally equivalent (about 0.36 mm) while the average knot pull force of the coated antimicrobial sutures of Example 2 are significantly better than the coated Vicryl® Plus. The average knot pull force of about 5.5 kgf or greater for the antimicrobial coated sutures of Example 2 is better than uncoated Polysorb® (+≈2%) and Vicryl Plus (+≈40%).

1-15. (canceled)

16. An antimicrobial coating composition for a surgical suture comprising:
a bioabsorbable polymer derived from one or more lactone monomers,
a fatty acid ester,
a rifamycin agent, and
a tetracycline agent.

17. The antimicrobial coating composition of claim 16, wherein the bioabsorbable polymer is derived from one or more lactone monomers selected from the group consisting of lactide, glycolide, trimethylene carbonate, caprolactone, and combinations thereof.

18. The antimicrobial coating composition of claim 16, wherein the bioabsorbable polymer derived from one or more lactone monomers includes a bioabsorbable copolymer derived include caprolactone and glycolide, wherein caprolactone represents a predominant amount of the bioabsorbable polymer.

19. The antimicrobial coating composition of claim 16, wherein the fatty acid ester is calcium stearoyl lactylate.

20. The antimicrobial coating composition of claim 16, wherein the rifamycin agent is rifampin and the tetracycline agent is minocycline.

21. The antimicrobial coating composition of claim 20, wherein the rifampin and the minocycline collectively represent a predominant component of the composition and the bioabsorbable polymer and the fatty acid ester collectively represent a minor component of the composition.

22. The antimicrobial coating composition of claim 21, further comprising one or more solvents selected from the group consisting of methylene chloride, ethanol, hexane, and combinations thereof.

23. An antimicrobial surgical suture comprising:
a suture made of a biocompatible suture material, and
an antimicrobial coating covering at least a portion of the suture, the antimicrobial coating including a minor amount of a bioabsorbable polymer derived from one or more first lactone monomers and a fatty acid ester, and a predominant amount of a rifamycin agent and a tetracycline agent.

24. The suture of claim 23, wherein the suture is a multifilament suture.

25. The suture of claim 23, wherein the biocompatible suture material comprises a bioabsorbable suture material including a polymer derived from one or more second lactone monomers.

26. The suture of claim 25, wherein the polymer derived from one or more second lactone monomers is a bioabsorbable copolymer derived from lactide and glycolide.

27. The suture of claim 26, wherein the bioabsorbable polymer derived from one or more first lactone monomers includes a bioabsorbable copolymer derived from caprolactone and glycolide, wherein caprolactone represents a predominant amount.

28. The suture of claim 23, wherein the fatty acid ester is calcium stearoyl lactylate.

29. The suture of claim 23, wherein the rifamycin agent is rifampin and the tetracycline agent is minocycline.

30. The suture of claim 29, wherein the antimicrobial coating further comprises alkylated rifampin, and the rifampin, the alkylated rifampin, and the minocycline each represent about 15-25 μg/mg of the suture.

31. The suture of claim 29, wherein the antimicrobial coating is sterile and includes about 30-50% alkylated rifampin.

32. The suture of claim 23, wherein the antimicrobial coating is configured to be a sustained release antimicrobial coating releasing the rifamycin agent and the tetracycline agent over 4 days and 21 days, respectively.

33. The suture of claim 23, wherein the antimicrobial coating produces at least a 4 log CFU reduction of S. aureus, E. coli, or MRSA in vivo.

34. A method of forming a coated antimicrobial suture comprising:
drawing an uncoated suture through an open container including an antimicrobial coating composition to form a wet coated suture, the antimicrobial coating composition including a bioabsorbable polymer derived from one or more lactone monomers, a fatty acid ester, a rifamycin agent, a tetracycline agent, and a solvent,
drawing the wet coated suture upwardly at a first angle from the open container and through a drying device to form a dry antimicrobial coated suture, and
drawing the dry coated suture downwardly at a second angle from an exit end of the drying device to a spool.
